(19) United States
(12) Patent Application Publication
(10) Pub. No.: US 2026/0217624 A1
(43) Pub. Date: Jul. 30, 2026

(54) FEEDSTOCK TO AROMATIC HYDROCARBONS CONVERSION VIA FORMALDEHYDE ROUTE IN DUAL BED REACTOR

(71) Applicant: KING ABDULLAH UNIVERSITY OF SCIENCE AND TECHNOLOGY, Thuwal (SA)

(72) Inventors: Teng LI, Thuwal (SA); Javier RUIZ-MARTINEZ, Thuwal (SA)

(21) Appl. No.: 19/142,687

(22) PCT Filed: Jan. 22, 2024
(86) PCT No.: PCT/IB2024/050602

§ 371 Date: Jun. 24, 2025

Related U.S. Application Data
(60) Provisional application No. 63/450,770, filed on Mar. 8, 2023.

(51) Int. Cl. C07C 1/20; B01J 8/04; B01J 23/06; B01J 29/40; C07C 1/207; C07C 2/76; C07C 45/29

(52) U.S. Cl. CPC C07C 1/20; B01J 8/0446; B01J 8/0492; B01J 23/06; B01J 29/405; C07C 1/2076; C07C 2/76; C07C 45/29; B01J 2208/00884; B01J 2208/025; C07C 2523/06; C07C 2529/40

(57) ABSTRACT

A catalytic kit for converting a feedstock to aromatics in a dual bed reactor includes a first catalyst A1 configured to promote (1) a conversion of a first part of the feedstock into olefins and (2) a conversion of diene and the olefines into the aromatics; a second catalyst A2 configured to promote a conversion of a second part of the feedstock into formaldehyde; and a third catalyst B configured to promote a conversion of olefines into additional aromatics, where the first and second catalysts A1, A2 are mixed together to be in direct contact with each other.

### CROSS-REFERENCE TO RELATED APPLICATIONS

This application claims priority to U.S. Provisional Patent Application No. 63/450,770, filed on Mar. 8, 2023, entitled “METHOD FOR PREPARING AROMATIC HYDROCARBONS THROUGH CONVERSION OF METHANOL VIA A FORMALDEHYDE ROUTE WITH CASCADE REACTIONS IN A DUAL BED,” the disclosure of which is incorporated herein by reference in its entirety.

### BACKGROUND OF THE INVENTION

### Technical Field

Embodiments of the subject matter disclosed herein generally relate to a system and method for converting a feedstock (e.g., methanol) to aromatic hydrocarbons while preserving the catalyst stability, and more particularly, to a dual bed reactor that is packed with a three-component catalyst kit for simultaneously providing aromatic selectivity and prevent catalyst deactivation.

### Discussion of the Background

Monocyclic aromatics or monoaromatics, in particular benzene, toluene and xylenes (BTX), are important building units for producing a myriad of chemicals such as plastics, resins, synthetic fibers, rubber lubricants, dyes, detergents, drugs, and pesticides. The conventional production method for monoaromatics follows a petroleum-based route, starting from an extracted fossil oil molecule, for example, following a distillation process of crude oil followed by naphtha reforming or steam cracking conversions. This route uses non-renewable raw materials, has a high energy consumption, and generates toxic by-products, which makes this technology undesirable. Thus, a new technology that is currently explored relies on the methanol-to-aromatic (MTA) process, which is a promising sustainable route to produce monoaromatics since methanol can be produced from biomass, waste and CO₂. The whole process involves methanol-to-olefins conversion (nCH₃OH═CₙH₂ₙ₊ₙH₂O) followed by olefins-to-aromatics conversion (CₙH₂ₙ₊₃CₘH₂ₘ═CₙH₂ₙ₋₆+3CₘH₂ₘ₊₂). In the MTA process, a zeolite catalyst is frequently used in combination with a metal, such as Ag, Ga and Zn. Both catalyst components work closely to drive the reaction toward aromatics, which involve a vast number of individual and sequential chemical reactions as exemplified in FIG. 1. However, this new technology faces low selectivity and inferior stability.

More specifically, FIG. 1 shows that a feed of methanol (M) molecules are dehydrated to dimethyl ether (D) molecules, which are further transformed into short-chain olefins (SCO) on Brønsted acid sites 110. The subsequent methylation and/or oligomerization leads to chain growth, producing long-chain olefins (LCO). Long-chain dienes (LCD) can be formed from LCO either by hydrogen transfer reactions on acids 110 forming inactive paraffins (P) simultaneously, or via direct dehydrogenation reactions imposed by metal sites 120 forming hydrogen gas in parallel. A subsequent cyclization of LCD leads to methylated cyclic species (MC), which can be transformed into monocyclic aromatics (MA) by hydride transfer reactions on acids 110 or metals 120. Acids 110 and metals 120 both contribute to the unwanted transformation of MA to polycyclic aromatics (PA). In addition, formaldehyde (F) can be produced on Brønsted acid sites 110 by methanol disproportionation or on aluminum-based Lewis acid sites 120 by hydrogen transfer from methanol to alkenes, both of which lead to inactive paraffins P. Methanol M can also be directly dehydrogenated on metal sites 120, producing hydrogen and formaldehyde F. Catalyzed by acids 110, the alkylation reactions between formaldehyde F and active MA via benzyl carbenium ions 130 produce diarylmethane 140, which can be rapidly converted into PA.

Current catalytic formulations maximize product selectivity by incorporating a large number of both active functionalities in individual catalyst particles with the aim of promoting each of the catalytic steps and the diffusion of intermediates from one active site to the next in the right sequence. Unfortunately, a high active-site density also promotes i) undesired hydrogen-transfer reaction leading to refractory paraffins P and ii) the formation of polycyclic aromatic PA or polyaromatic species causing catalyst deactivation. Improving molecular diffusivity by the formation of mesoporous hierarchical structures or nanosize zeolites increases catalyst lifetime but significantly decreases product selectivity. This status of the art indicates that the task of producing monoaromatics efficiently and mitigating side reactions and catalyst deactivation is challenging because of the incompatibility between aromatic selectivity and catalyst stability.

Thus, there is a need for a new catalyst configuration and a new path for transforming the methanol into monocyclic aromatics that preserves the aromatic selectivity (i.e., formation of MA and not PA) and maintains the catalyst (acid or metal) stability during the process.

### SUMMARY OF THE INVENTION

According to an embodiment, there is a catalytic kit for converting a feedstock to aromatics in a dual bed reactor. The catalytic kit includes a first catalyst A1 configured to promote (1) a conversion of a first part of the feedstock into olefins and (2) a conversion of diene and the olefines into the aromatics, a second catalyst A2 configured to promote a conversion of a second part of the feedstock into formaldehyde, and a third catalyst B configured to promote a conversion of olefines into additional aromatics. The first and second catalysts A1, A2 are mixed together to be in direct contact with each other.

According to another embodiment, there is a reactor configured for converting a feedstock to aromatics. The reactor includes a housing divided into an upper chamber and a bottom chamber by a porous divider, an inlet configured to receive the feedstock, the inlet being in fluid communication with the upper chamber, an outlet configured to discharge the aromatics from the bottom chamber, and a catalyst kit having first and second catalysts A1 and A2 distributed in the upper chamber and a third catalyst B distributed in the bottom chamber. The first catalyst A1 is configured to promote (1) a conversion of a first part of the feedstock into olefins, and (2) a conversion of diene and a first part of the olefines into the aromatics, the second catalyst A2 is configured to promote a conversion of a second part of the feedstock into formaldehyde, and the third catalyst B is configured to promote a conversion of a second part of the olefines into additional aromatics, and the first and second catalysts A1, A2 are mixed together to be in direct contact with each other.

According to yet another embodiment, there is a method for converting a methanol feed to aromatics, and the method includes receiving a catalyst kit having first and second catalysts A1 and A2, and a third catalyst B, wherein each of the three catalysts is different from the other two catalysts, loading the first and second catalysts A1 and A2 in an upper chamber of a reactor, wherein the first catalyst A1 is configured to promote (1) a conversion of a first part of the methanol feed into olefins, and (2) a conversion of dienes and a first part of the olefines into the aromatics, and the second catalyst A2 is configured to promote a conversion of a second part of the methanol feed into formaldehyde, loading the third catalyst B in a bottom chamber of the reactor, wherein the third catalyst B is configured to promote a conversion of a second part of the olefines into additional aromatics, supplying the methanol feed to the upper chamber of the reactor, and converting the methanol feed to the aromatics in each of the upper and bottom chambers. The first and second catalysts A1, A2 are mixed together to be in direct contact with each other in the upper chamber.

### BRIEF DESCRIPTION OF THE DRAWINGS

For a more complete understanding of the present invention, reference is now made to the following descriptions taken in conjunction with the accompanying drawings, in which:

FIG. 1 is a schematic diagram of possible paths for transforming methanol into aromatics in the presence of acid and metal sites catalysts;

FIGS. 2A to 2C illustrate various catalysts packing modes, with FIG. 2A showing a catalyst that contains zeolite (named HZ140) only, FIG. 2B showing a catalyst that contains zeolite packed below ZnO, and separated by an inert layer of quartz wool of about ~3 mm in thickness, and FIG. 2C showing the zeolite and ZnO mixed together;

FIGS. 3A to 3C show reaction results with time-on-stream (TOS) over the catalysts packed as illustrated in FIGS. 2A to 2C, respectively;

FIGS. 4 and 5 schematically illustrate the methanol conversion to monocyclic aromatics over an A1 catalyst;

FIGS. 6A and 6B schematically illustrate the methanol conversion to monocyclic aromatics over a mixture A=A1/A2 catalyst;

FIG. 7A schematically illustrates a double bed reactor having a catalyst kit distributed in the two chambers of the reactor;

FIG. 7B schematically illustrates the chemical reaction taking place in each chamber of the reactor and the distribution of the catalysts of the three-component catalyst kit inside the reactor;

FIG. 8A illustrates the conversion results over different catalysts, FIG. 8B illustrates the selectivity to aromatics for the different catalysts from FIG. 8A, and FIG. 8C illustrates the aromatics distribution for each catalyst;

FIG. 9 illustrates a comparison between a three-component catalyst kit and traditional catalysts used for both aromatic selectivity and catalyst lifetime for the methanol-to-aromatic process; and

FIG. 10 is a flow chart of a method for converting methanol to aromatics with a three-component catalyst kit that extends the life of the catalyst and prevents the formation of polycyclic aromatics.

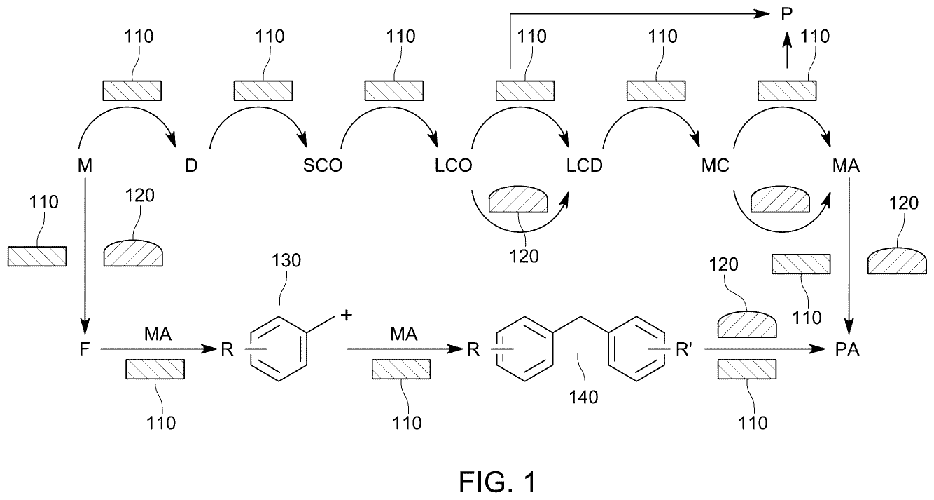

### DETAILED DESCRIPTION OF THE INVENTION

The following description of the embodiments refers to the accompanying drawings. The same reference numbers in different drawings identify the same or similar elements. The following detailed description does not limit the invention. Instead, the scope of the invention is defined by the appended claims. The following embodiments are discussed, for simplicity, with regard to the methanol conversion using a catalyst kit that includes at least three different catalysts, located in a dual bed reactor. However, the embodiments to be discussed next are not limited to the specific three component catalyst kit analyzed herein, but may be applied to other catalyst kits that achieve the functionality of the described kit, and the kit may include more than three catalysts. Moreover, the feedstock is not limited to methanol, but may include other alcohols (such as ethanol, propanol, butanol and isobutanol), olefins (such as ethene, propene, butene, pentene), as well as the mixture of olefins and alcohols mentioned above. For simplicity, the following embodiments are discussed with a feedstock that includes only methanol. One skilled in the art would understand that the same principles apply if the feedstock is one of the other alcohols or olefines mentioned above.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

It will be understood that, although the terms first, second, etc. may be used herein to describe various elements, these elements should not be limited by these terms. These terms are only used to distinguish one element from another. For example, a first object or step could be termed a second object or step, and, similarly, a second object or step could be termed a first object or step, without departing from the scope of the present disclosure. The first object or step, and the second object or step, are both, objects or steps, respectively, but they are not to be considered the same object or step.

The terminology used in the description herein is for the purpose of describing particular embodiments and is not intended to be limiting. As used in this description and the appended claims, the singular forms “a,” “an” and “the” are intended to include the plural forms as well, unless the context clearly indicates otherwise. It will also be understood that the term “and/or” as used herein refers to and encompasses any possible combinations of one or more of the associated listed items. It will be further understood that the terms “includes,” “including,” “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Further, as used herein, the term “if” may be construed to mean “when” or “upon” or “in response to determining” or “in response to detecting,” depending on the context.

According to an embodiment, a novel three-component catalyst kit is used for controlling the chemistry of a different conversion path of the methanol into aromatics. In this embodiment, the methanol is first selectively converted into formaldehyde and light olefins (note that the wisdom in the art is to try to avoid the formation of formaldehyde by decreasing the amount of active sites for its generation or by accelerating its decomposition by adding some additives). This step goes against this wisdom and on purpose produces formaldehyde. Two different catalysts are used for this step, a first catalyst A1 for promoting the formation of the olefines and a second catalyst A2 for promoting the formation of the formaldehyde. The first catalyst is selected to have a low number of acid sites, e.g., for a catalyst that includes Si and Al atoms, a ratio of the Si/Al is between 50 and 1000 (including 50) for achieving the “low” number of acid sites. Because of the low number of acid sites, the formaldehyde formation is hindered. To increase the formation of the formaldehyde from the methanol feed in the same chamber/bed of the reactor, the second catalyst A2 is selected to promote formaldehyde formation. Further, the two catalysts are intimately mixed together in this chamber of the reactor. As a consequence of this selection, the formaldehyde interacts with the formed olefine molecules and results in dienes via Prins and/or Diels-Alder reactions. High selectivity (i.e., forming MA and not PA) is achieved by the precise location of the active sites of different nature in the dual bed reactor. The first catalyst A1 was also selected to promote the reaction of dienes with the remaining olefines to form monocyclic aromatics. All these reactions take place in the upper chamber of the reactor, where the mixture of the A1 and A2 catalysts is provided.

The monocyclic aromatics and some of the left-over olefines enter then into the bottom chamber/bed of the reactor, where a third catalyst B is provided. The catalyst B is selected to transform the remaining olefines into the monocyclic aromatics, i.e., it has a high number of acid sites, for example, for a catalyst that includes Si and Al atoms, a ratio of the Si/Al is between 10 and 50 (excluding 50) for achieving the “high” number of acid sites. Note that essentially no methanol and no formaldehyde (practically, some traces of these molecules may be found in the second chamber of the reactor, but they are insubstantial, e.g., less than 5% of the total molecules by mass entering the second chamber) enter the second chamber of the reactor, and thus, the catalyst B can be selected to fully transform the remaining olefines into the monocyclic aromatics.

The selection of the three-component catalyst kit discussed above not only has a high rate of conversion of the methanol into the aromatics, but also results in a very stable catalyst as the low number of acid sites in A1 prevent its deactivation and the high number of acid sites in the catalyst B do not inactivate it because no formaldehyde is present in the second chamber of the reactor. The selection of this three-component catalyst kit for distribution in two different chambers of the reactor is now discussed in more detail.

The catalyst kit, which is a combination of inexpensive and commercially available individual catalysts, may include, according to this embodiment, the following components: (1) catalyst A1=HZSM-5, (2) catalyst A2=ZnO, and (3) a catalyst N=Zn-ion exchanged/modified HZSM-5. This kit may achieve an initial aromatic selectivity of 71.4%, and above 40% after a 400-h test with full methanol conversion. In addition, the formed monocyclic aromatics make up nearly all (>98%) of the liquid products, and the BTX (benzene, toluene, and xylenes) account for 91% of the monoaromatics, which is advantageous for product separation. According to this novel strategy, it is possible to balance product selectivity and catalyst stability in complex heterogeneous catalytic systems. Note that HZSM-5, which stands for Zeolite Socony Mobil-5, is an aluminosilicate zeolite belonging to the pentasil family of zeolites. Its chemical formula is NaₙAlₙSi₉₆₋ₙO₁₉₂·16H₂O (0<n<27).

A challenge for the selective formation of monoaromatics from methanol lies in the precise control of the reactions that lead to monoaromatics while suppressing hydrogen-transfer reactions and aromatic condensation into larger deactivating species. The inventors have discovered that an alternative chemical pathway based on the intentional production of formaldehyde might circumvent hydrogen-transfer reactions and be an important precursor of monoaromatics. Because formaldehyde is responsible for catalyst deactivation in the traditional approach, a careful control of the catalytic reactions is required by the design of the nature and number of metal and acid sites in a reactor, as now discussed.

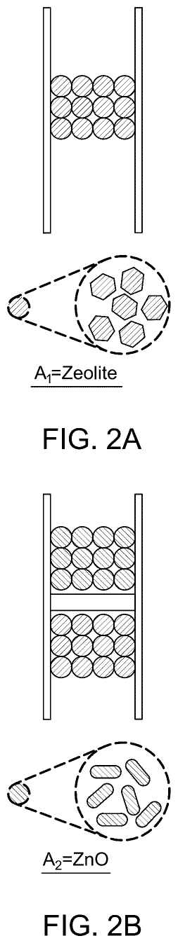

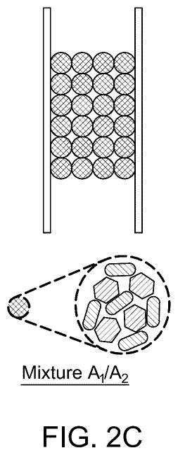

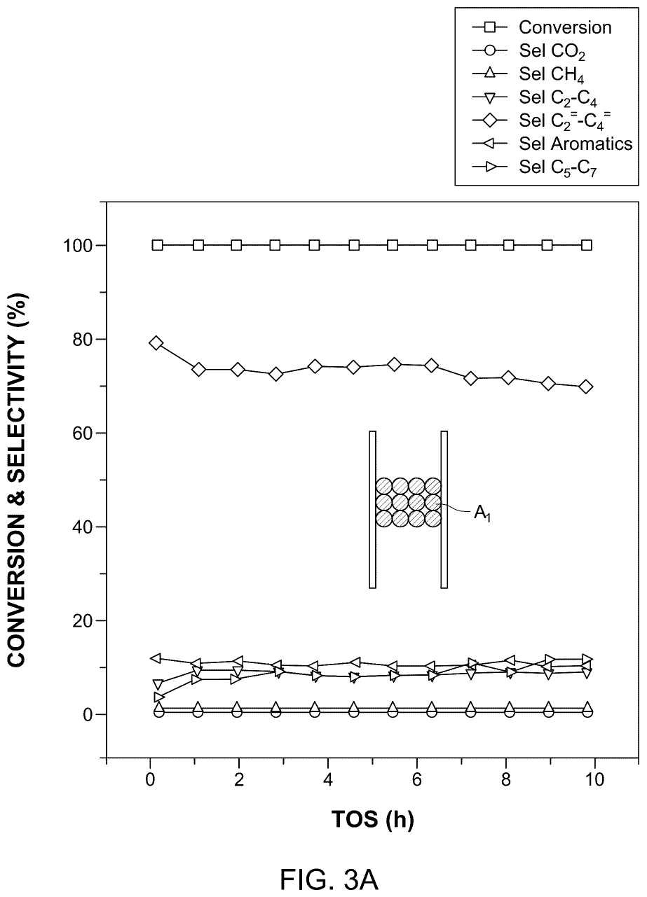

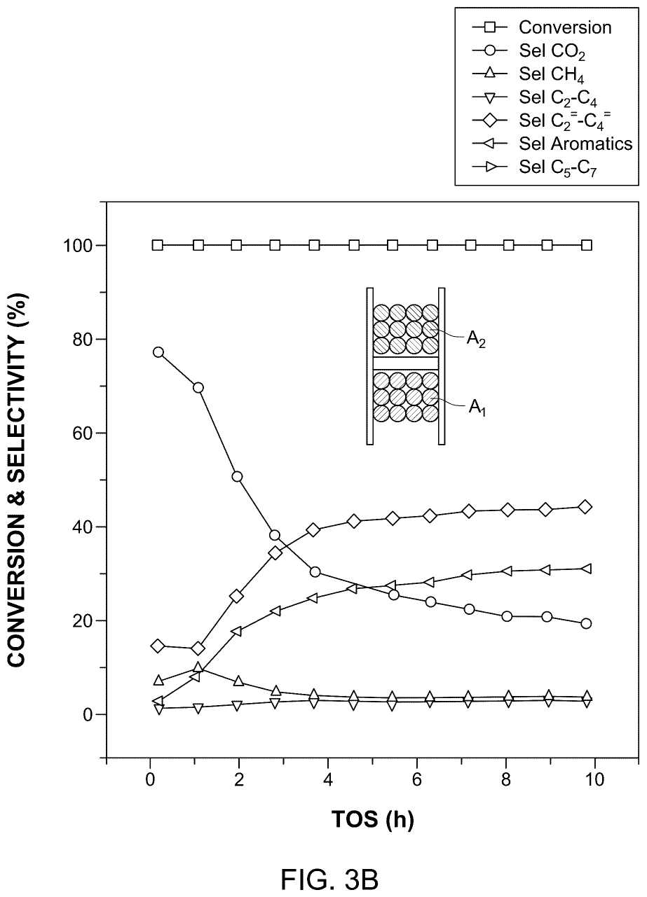

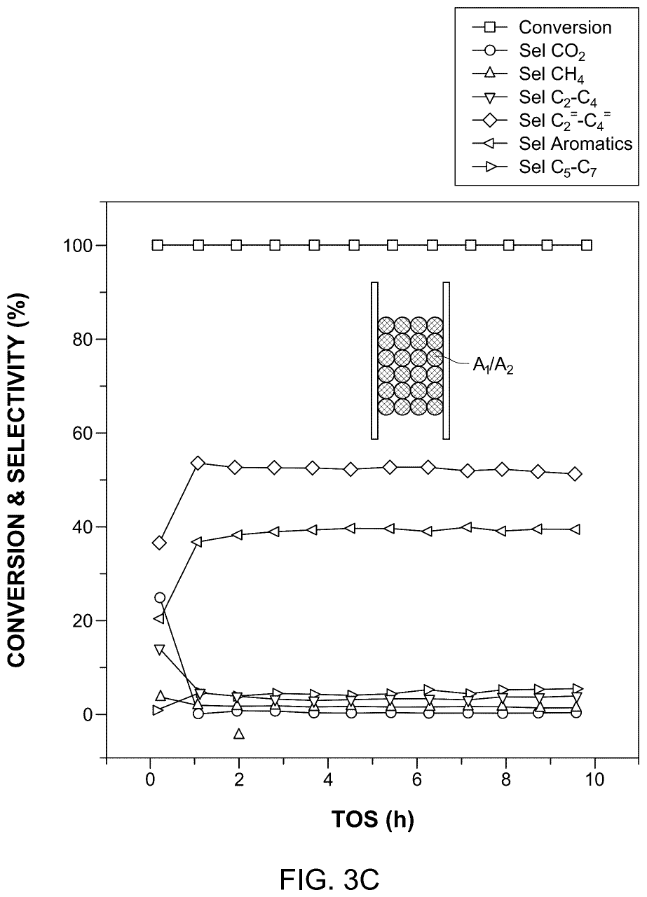

Initially, the inventors have used a catalyst kit containing A1=HZSM-5 zeolite with a low number of acid sites (HZ140, Si/Al ratio of 140) and A2=metal oxide (ZnO) that is able to produce some formaldehyde under typical MTA reaction conditions. The two components were packed in different modes as shown in FIGS. 2A to 2C. The zeolite component mainly contains crystals larger than 1 μm, while the ZnO component consists of rod-shaped crystals of different sizes with the largest dimension being around 400 nm. FIG. 3A shows that the standalone zeolite A1 was very stable and reached around 70% selectivity to C2-C4 olefins, with a very modest selectivity to aromatics (around 10%). In the next step, a layer of ZnO A2 was added on top of the zeolite A1 to convert a fraction of methanol into formaldehyde. In such configuration, there was an increase in the aromatic selectivity to around 30% upon about 2 h induction period. When the ZnO and the zeolite were physically mixed in a mortar (FIG. 3C), the production of monoaromatics was stable with an average selectivity close to 40%. It is also worth to mention that paraffin selectivity was kept very low (3%). Paraffins are undesired products as generally have low reactivity and require high temperatures to be dehydrogenated to olefins and then converted into aromatics. The inventors have observed that the composite catalyst A=A1/A2 (ZnO+HZ140) was very stable, yielding full methanol conversion in the investigated timespan.

To confirm that formaldehyde (HCHO) is formed during the reaction, the inventors monitored it in situ by using planar laser-induced fluorescence (PLIF) imaging as formaldehyde analysis is challenging with normal online analytical tools. For the measurements, the reactive intermediates formed in the gas phase close to the surface of the catalyst bed were monitored by PLIF (not shown). When methanol was first fed into the reactor, formaldehyde was not observed on ZnO, most likely due to the complete dehydrogenation to CO and H2. With the gradual deactivation of ZnO, formaldehyde concentration started to increase. HZ140 only yielded HCHO at the beginning when methanol was fed. However, the HCHO signal over mixture was much lower than over pure ZnO, indicating that in-situ generated HCHO molecules are consumed, most likely after reaction with olefins forming aromatics.

Because ZnO could have other catalytic properties than the production of formaldehyde, control experiments were performed to fully understand the role of ZnO in the reaction. Zn-containing zeolites have been widely explored in the methanol-to-aromatic reaction for their olefin dehydrogenation properties, resulting in the formation of dienes and aromatics. To evaluate this effect, the catalysts were tested in the conversion of propylene. As compared to using methanol as a feedstock, ZnO+HZ140 displayed only 2% aromatic selectivity in propylene conversion. This indicates that the limited dehydrogenation capabilities of ZnO particles did not contribute significantly to olefin aromatization and the key is in the methanol conversion.

After confirming the formation of formaldehyde, the inventors investigated its role by cofeeding it in the absence of ZnO. The investigation started with the use of formalin/methanol mixtures. As formalin contains water and this can have an effect on the reaction, a control experiment was done by using methanol/water mixtures. Comparing methanol/water and methanol/formalin as feedstocks illustrates the promoting effect of HCHO. However, the increase in selectivity, from 10.2% to 16.3%, was less pronounced than using pure methanol. A possible reason is the large amount of water present in formalin. In contrast, dimethoxymethane (DMM), which equally decomposes into dimethyl ether and formaldehyde without water formation, led to a higher aromatic selectivity (57.5%). Nevertheless, the catalysts lifetime was shortened, most likely because of the high concentration of HCHO during DMM decomposition, leading to the formation of deactivating polyaromatic species.

Next, the inventors cofed ¹³C labeled formalin with methanol and tracked the species containing ¹³C. The reaction pathway of ¹³C-labeled formaldehyde was traced by a traditional mass spectrometry (MS) employing electron impact ionization at 70 eV. The results show that increasing the percentage of HCHO allows the formation of more ¹³C-labeled butadiene, an intermediate reaction in the formation of aromatics. A possible explanation is that HCHO reacts with olefins via Prins reaction into dienes. As a result, hydrogen transfer reactions of olefins to form dienes and paraffins are suppressed.

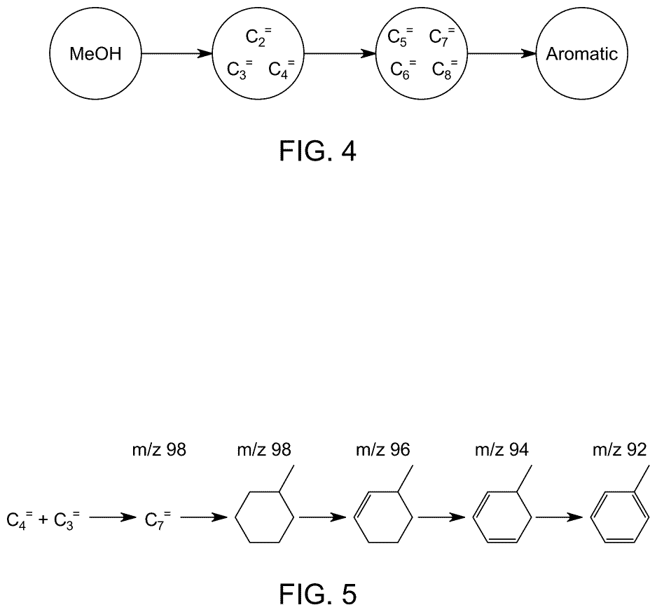

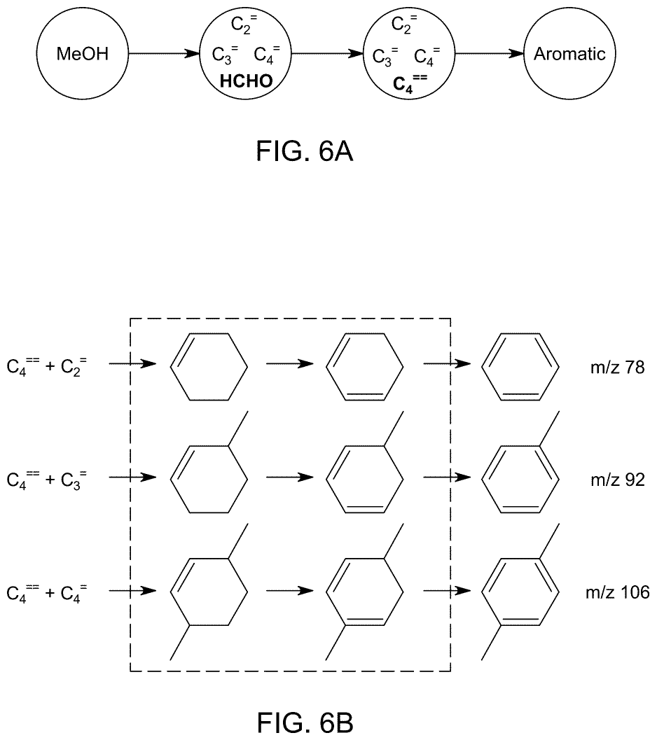

To better describe how the composite catalyst A1/A2 (e.g., ZnO+HZ140) steers the methanol conversion towards the desired monocyclic aromatics, the reaction intermediates and products were investigated. Using toluene as an example, over HZ140, dimerization between propylene and butene (dominant products) could lead to heptene, which then undergoes cyclization (one step) and hydrogen transfer reactions (three steps) to form toluene, as schematically illustrated in FIGS. 4 and 5. The low density of acid sites in HZ140 somehow slows down the whole progress. Over ZnO+HZ140, it is believed that the in-situ generated HCHO reacts with propylene to yield butadiene first, which has also been observed in the isotopic labeling experiments. Then, butadiene reacts with another propylene via Diels-Alder reaction to form methyl cyclohexene. The improved dehydrogenation ability, either from ZnO particles or newly formed Zn²⁺ species, could help the transformation of methyl cyclohexene to toluene, as illustrated in FIGS. 6A and 6B. By involving highly active molecules, such as formaldehyde and butadiene, in serial cascade reactions, the reaction proceeds downhill and thus has a lower dependency on the number of acid sites. As a result, reactions yielding side products (paraffins and polycyclic aromatics) are suppressed, while stable and efficient production of monocyclic aromatics is obtained.

Characterization of species trapped in the catalyst by ¹³C solid-state nuclear magnetic resonance (ssNMR) spectroscopy provide complementary information supporting these findings. Over ZnO, in addition to the formate, the dominant deposit species were methylated conjugated polyenes. In contrast, besides the formate, the deposit species were mainly methylated aromatics over mixture. Such a difference suggests that the generated HCHO molecules can undergo different evolutions depending on the presence and absence of zeolite.

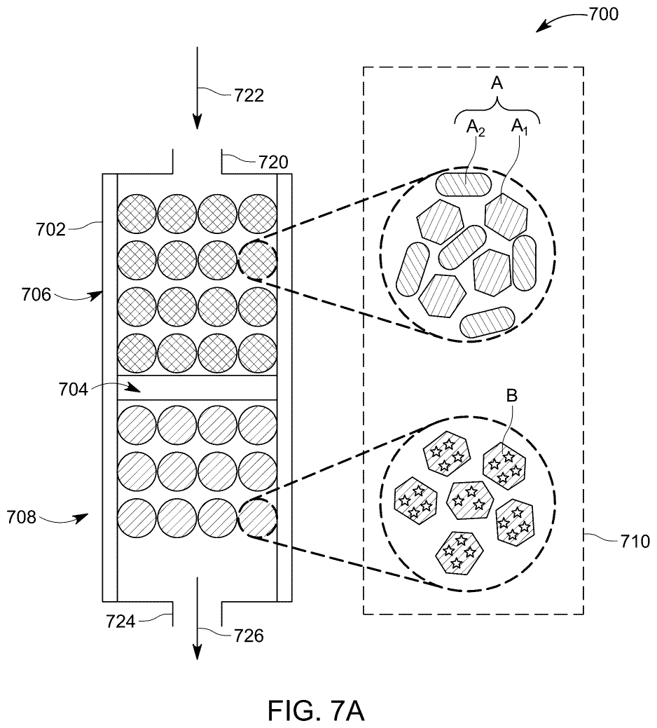

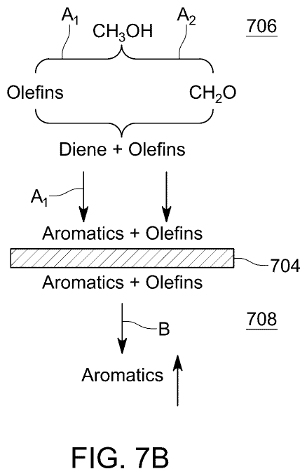

Catalysis relay (i.e., the usage of plural catalysts in the same reactor with a product experiencing multiple interactions with the catalysts along its chemical path) can further increase the selectivity of aromatics. As light olefins are the main species in the effluent from the upper chamber, aside from the monocyclic aromatics, it is believed that they can be further transformed by adding another layer of catalyst (third catalyst, B) in the bottom chamber, as illustrated in FIG. 7A. FIG. 7A shows a reactor 700 having a housing 702 (made, for example, of quartz) that is divided by a divider 704, e.g., quartz wool, into upper/top and bottom chambers, for forming the dual bed reactor. The dual bed reactor 700 has an upper chamber 706 and a lower chamber 708, which are separated from each other by the divider 704. Note that a gas/product/effluent from the upper chamber passes the divider 704 to enter the bottom chamber as the divider is porous. The first catalyst A (i.e., mixture A1/A2), which includes catalyst A1 and catalyst A2 is placed in the upper chamber 706 while the second catalyst B is placed in the lower chamber 708. The housing 702 may have an inlet 720 for receiving a methanol feed 722 and the inlet fluidly and directly communicates with the upper chamber 706. The housing 702 may also have an outlet 724 for discharging the produced aromatics 726 and the outlet fluidly and directly communicates with the bottom chamber 708.

In one embodiment, assuming the reactor 700's volume is 100%, the quartz wool (component is SiO₂) divider 704 occupies 5~10% volume of the reactor. It is placed in the middle layer used to isolate the two chambers, avoiding the direct contact between catalyst A (i.e., mixture A1/A2) and B. Catalyst A occupies about 20~40% of the volume of the entire reactor). In one application, the catalyst A is a powder mixture of the zinc oxide (ZnO) and the H-ZSM-5 zeolite with a Si/Al ratio of 50~1000. The mass ratio between ZnO and the zeolite is 1:1, with a range of 0.1~10. Catalyst B may occupy about 20%~50% of the entire volume of the reactor. Catalyst B may be a zinc-modified H-ZSM-5 zeolite. In one application, the catalyst B has a Si/Al ratio of 10~50, and the zinc loading is 1~5 wt %. Those skilled in the art will understand that the numbers provided herein are for reference only and these numbers can vary by up to 20% and still obtain the same results. The components of catalyst A in this embodiment are H-ZSM-5 zeolites from Zeolyst, and Zinc Oxide from Sigma-Aldrich. Catalyst B may be prepared by the ion-exchanging method. For example, 1 gram of ammonium-form ZSM-5 is placed in a 40 ml 0.1 M zinc nitrate solution, stirred at 50° C. for 6 hours, and then washed with deionized water thoroughly; the catalyst is then dried at 80° C. for 12 hours and calcined at 550° C. for 6 hours. Other methods for making catalyst B may be used.

In one application, the catalyst A1 is one of Silicon oxide (SiO₂), Magnesium oxide (MgO), ZSM-5 or H-ZSM-5 (MFI topology, which is specific structure type, i.e., silicalite-1); ZSM-11 or H-ZSM-11 (MEL topology, which is described in Ch. Baerlocher, WM Meier, DH Olson, Atlas of Zeolite Framework Types, 5th Edition, 2001); ZSM-22 or H-ZSM-22 (TON topology, which is another possible topology); MCM-22 or H-MCM-22 (MWW topology, which is another possible topology); SSZ-74 or H-ZSM-22 (SVR topology); or SSZ-35 or H-SSZ-35 (STF topology). A common feature of all the catalysts A1 is the promotion of the formation of olefins from the methanol and also the promotion of the reaction between olefins and dienes to form monocyclic aromatics.

The catalyst A2 is one of Zinc oxide (ZnO); Vanadium oxide (V₂O₅); Chromium oxide (Cr₂O₃); Magnesium oxide (MgO); Iron oxide (Fe₃O₄); Cobalt oxide (Co₃O₄); Nickel oxide (NiO); Copper oxide (CuO); Gallium oxide (Ga₂O₃); silver oxide (Ag₂O); Indium oxide (In₂O₃); Molybdenum oxide (MoO₃); or Tungsten oxide (WO₃). A common feature of all the catalysts A2 is the promotion of the formation of formaldehyde from methanol.

The catalyst B is one of Zinc-modified H-ZSM-5 or H-ZSM-11 or H-ZSM-22 or H-MCM-22 or H-ZSM-22 or H-SSZ-35; or Vanadium-modified H-ZSM-5 or H-ZSM-11 or H-ZSM-22 or H-MCM-22 or H-ZSM-22 or H-SSZ-35; or Chromium-modified H-ZSM-5 or H-ZSM-11 or H-ZSM-22 or H-MCM-22 or H-ZSM-22 or H-SSZ-35; or Magnesium-modified H-ZSM-5 or H-ZSM-11 or H-ZSM-22 or H-MCM-22 or H-ZSM-22 or H-SSZ-35; or Iron-modified H-ZSM-5 or H-ZSM-11 or H-ZSM-22 or H-MCM-22 or H-ZSM-22 or H-SSZ-35; or Cobalt-modified H-ZSM-5 or H-ZSM-11 or H-ZSM-22 or H-MCM-22 or H-ZSM-22 or H-SSZ-35; or Nickel-modified H-ZSM-5 or H-ZSM-11 or H-ZSM-22 or H-MCM-22 or H-ZSM-22 or H-SSZ-35; or Copper-modified H-ZSM-5 or H-ZSM-11 or H-ZSM-22 or H-MCM-22 or H-ZSM-22 or H-SSZ-35; or Gallium-modified H-ZSM-5 or H-ZSM-11 or H-ZSM-22 or H-MCM-22 or H-ZSM-22 or H-SSZ-35; or Silver-modified H-ZSM-5 or H-ZSM-11 or H-ZSM-22 or H-MCM-22 or H-ZSM-22 or H-SSZ-35; or Indium-modified H-ZSM-5 or H-ZSM-11 or H-ZSM-22 or H-MCM-22 or H-ZSM-22 or H-SSZ-35; or Molybdenum-modified H-ZSM-5 or H-ZSM-11 or H-ZSM-22 or H-MCM-22 or H-ZSM-22 or H-SSZ-35; or Tungsten-modified H-ZSM-5 or H-ZSM-11 or H-ZSM-22 or H-MCM-22 or H-ZSM-22 or H-SSZ-35. A common feature of all possible catalysts B is the promotion of the olefins to aromatics transformation.

In this embodiment, when the reactor 700 was packed with a three-component catalyst kit 710 that includes A1=H-ZSM-5 zeolite with Si/Al of 50~1000, A2=ZnO, and B=zinc-modified H-ZSM-5 zeolite with Si/Al smaller than 50, with A1 and A2 being located in the upper chamber and in direct contact with each other, and B located in the bottom chamber, it was found that the upper chamber converts a first part of the methanol to olefins due to A1 and a second part of the methanol to formaldehyde due to A2, and the formed formaldehyde is then transformed to dienes due to the interaction with some of the olefins. Still in the upper chamber, the dienes are transformed into monocyclic aromatics due to the catalyst A1 so that only aromatics and olefines pass the divider 704 and enter the bottom chamber 708. While in the bottom chamber 708, the remaining olefines are transformed into monocyclic aromatics due to the catalyst B, as schematically illustrated in FIG. 7B. As a result, the product selectivity of the catalyst kit 710 in the dual bed reactor 700 is above 80%, and the reaction can run more than 500 hours without catalyst deactivation, showing large industrial application potential.

The inventors have found that in the upper chamber 706, 10~20% of methanol molecules are converted to formaldehyde on ZnO and 80~90% of methanol molecules are converted to light olefins on H-ZSM-5 for this specific selection of the catalyst kit 710. This is achieved by flowing methanol to the mixture of ZnO and H-ZSM-5. Keeping the two catalyst components close (e.g., in direct contact) to each other aids the timely transfer of the intermediate formaldehyde, from the ZnO part to the zeolite part, to finally yield aromatics. Separating the two components would lead to the decomposition of formaldehyde into valueless CO on ZnO. The reaction between formaldehyde and olefins leads to aromatics products (taking up 40% of the whole products), and also produces less unreactive paraffins (taking up 3% of the whole products). Due to the high Si/Al ratio of the zeolite component (Si/Al>100, for example, 140) in the catalyst A, the coke formation rate is pretty low, leading to a long catalyst lifetime (>150 h).

In the bottom chamber 708, where all products from the upper bed flow in, the inventors found that the zinc-modified HZSM-5 could effectively convert the remaining olefins into aromatics. Meanwhile, due to the lack of formaldehyde in the bottom chamber, the catalyst B is not deactivated. For this embodiment, the weight hourly space velocity of methanol was 2 Kg methanol/Kg catalyst/h. Other velocities may be used for the methanol feed, depending on the size of the reactor. The mass ratio between catalyst A and catalyst B is about 1:1 in this embodiment. The term “above” is used herein as meaning a variation of ±20% of the quantity characterized by this term. The feedstock may be a mixture of methanol and N₂ (molar ratio=1:3). The reaction in the reactor 700 may be operated at atmospheric pressure (1 bar) in a temperature range of about 400~500° C. One skilled in the art would understand that these numbers can be varied up to 30% and still achieve the advantages discussed herein.

The method of combining the above noted selection of catalysts and a zoned reactor leads to >500 h catalyst lifetime. The final aromatic products take up above 80% of the total products, and 91% of these aromatics are valuable BTX. The selection of the catalyst A in the upper chamber can include many mixtures of two components, of which one is responsible for formaldehyde formation and the other is for olefins formation. The selection of the catalyst B in the bottom chamber can include many zeolites or metal-modified zeolites that can selectively convert light olefins into aromatics.

The absence of HCHO or methanol (HCHO precursor) in the bottom chamber 708 allowed the inventors to follow a traditional strategy to produce aromatics in this bottom chamber, by incorporating a large number of active metal and acid sites in individual catalyst particles. Both H-form and Zn-form zeolites were tested as catalyst B. H-form zeolites were prepared by calcining commercial zeolites of various Si/Al ratios, and Zn-form zeolites were obtained by ion-exchanging parent zeolites with zinc nitrate solutions followed by calcination. Using ZnHZ12 with the highest A1 content (Si/Al=12) and the highest Zn loading (3.5 wt. %) resulted in the most satisfactory performance.

Energy dispersive X-ray (EDX) spectroscopy map of the B catalyst ZnHZ12 indicates that the introduced Zn species are homogeneously distributed. The replacement of protons by zinc cations enhances the dehydrogenation capacity, as demonstrated in propane dehydrogenation reactions. Furthermore, such Zn distribution also shortens the distance between metal sites and acid sites.

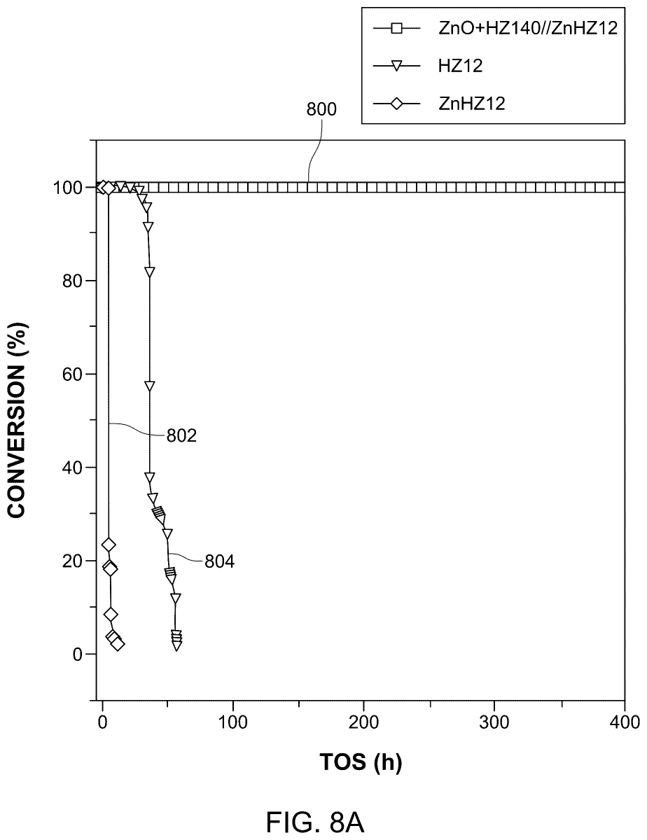

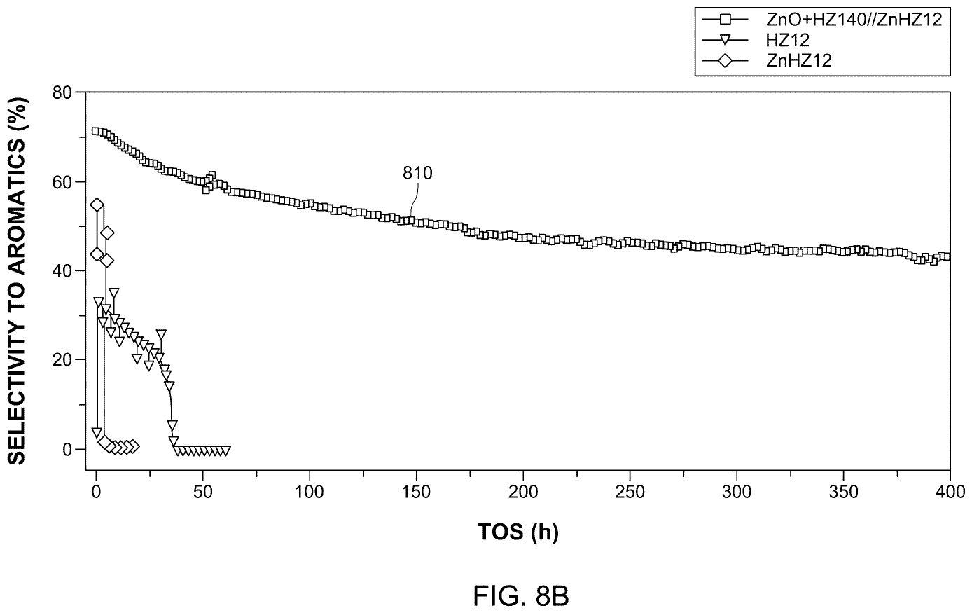

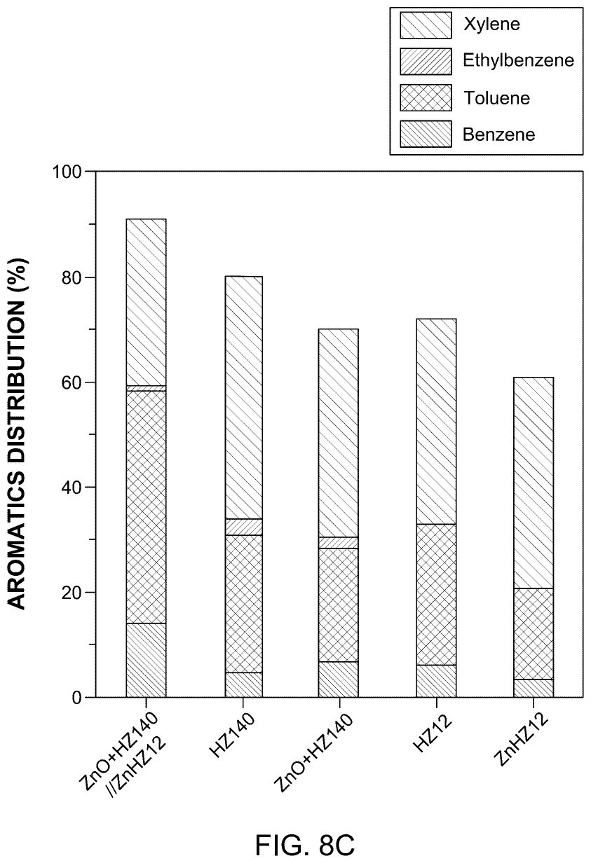

The performance of the composite catalyst kit 710 that includes the components A1=HZ140, A2=ZnO, and B=ZnHZ12, is illustrated in FIGS. 8A to 8C. For comparison, the H-form HZ12 and its Zn-exchanged analogues ZnHZ12 were also tested. After catalysis relay, the initial selectivity to monocyclic aromatics over ZnO+HZ140//ZnHZ23 was enhanced to 71.4%. In contrast, the highest aromatic selectivity over HZ12 and ZnHZ12 was 34.9% and 54.8%, respectively. The composite catalysts also gave satisfactory product distribution. In the 10-h test, the average selectivity for monocyclic aromatics, C2-C4 olefins, C2-C4 paraffins, C5-C7 hydrocarbons, and methane are 70.4%, 12.7%, 12.8%, 0.8%, and 3.4%, respectively. When considering the selectivities in the liquid phase, the results are even more relevant where aromatic selectivity is 98.8%, which is a strong competitive advantage for a potential industrial applications as separation cost will be drastically diminished.

The stability of the ZnO+HZ140//ZnHZ12 catalyst kit is outstanding compared to previous studies. In the 400-h test, the inventors did not observe any deactivation of the catalysts, as illustrated by curve 800 in FIG. 8A. On the contrary, the methanol conversions had dropped below 50% after 35 h for HZ12 (see curve 802) and 4 h for the reaction with ZnHZ12, (see curve 804), respectively, as also shown in FIG. 8A. The short lifetime of HZ12 and ZnHZ12 can be explained by the coincidence of formaldehyde and active sites both in large quantities, which accelerates the formation of polycyclic aromatics. The selectivity to aromatics over these two samples also declined rapidly with TOS, as illustrated in FIG. 8B. In contrast, the selectivity over ZnO+HZ140//ZnHZ23 declined slowly (see curve 810 in FIG. 8B), and the value was above 40% for the entire test period. Furthermore, the distribution of aromatic species had shifted to producing more lighter arene molecules, as illustrated in FIG. 8C. Specifically, the percentage of BTX in total monocyclic aromatics was 91% for the catalyst kit 710, while this value was only 72% for HZ12 and 61% for ZnHZ12, respectively. More benzene (15.4%) and toluene (48.6%) were produced as well with the catalyst kit 710. Note that the weight of each catalytic component for the above tests was: HZ140, 20 mg; ZnO, 20 mg; ZnHZ12, 40 mg and the reaction conditions were: 450° C.; MeOH rate, 0.04 g h-1; MeOH/N2 molar ratio, 1:3; and pressure inside the reactor was 0.1 MPa.

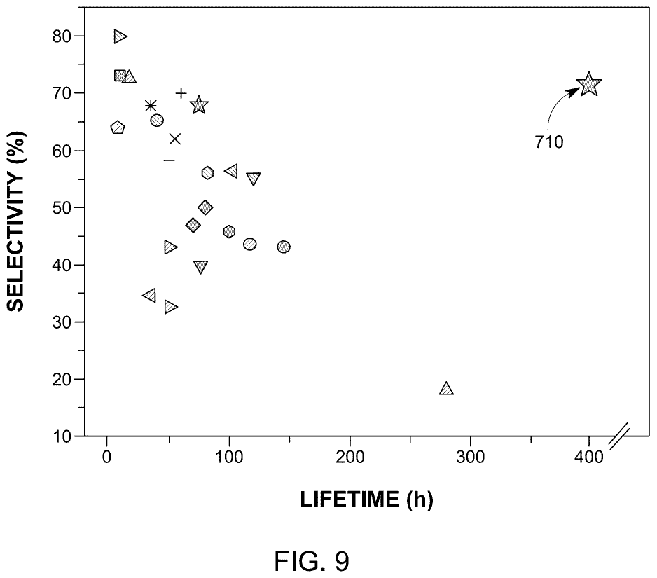

These results demonstrate that the chemistry for methanol conversion to aromatic can be controlled properly by precisely locating active sites of different nature (metals and acid sites) in a two-bed reactor by using the novel catalyst kit 710. Comparisons with state-of-the-art catalysts prove that this strategy is feasible, since it shows better performance in both selectivity and stability, as shown in FIG. 9. In addition, the catalysis relay discussed herein may be applied in other processes, for example, in the conversion of alcohols or olefins to produce aromatics, in which both product selectivity and catalyst stability should simultaneously be considered.

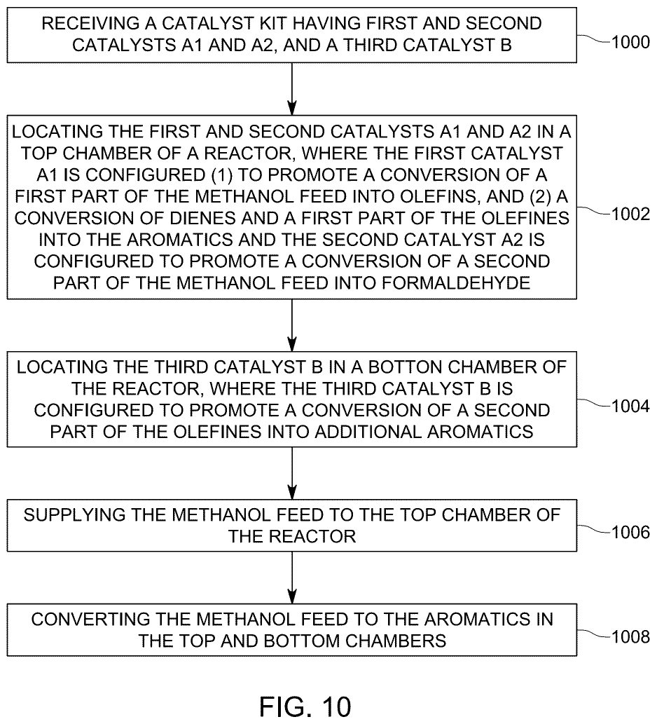

A method for converting the methanol feed 722 to aromatics 726 is now discussed with regard to FIG. 10. The method includes a step 1000 of receiving a catalyst kit having first and second catalysts A1 and A2, and a third catalyst B, a step 1002 of locating the first and second catalysts A1 and A2 in a upper chamber of a reactor, where the first catalyst A1 is configured to promote a conversion of a first part of the methanol feed into olefins, and the second catalyst A2 is configured to promote (1) a conversion of a second part of the methanol feed into formaldehyde, and (2) a conversion of dienes and a first part of the olefines into the aromatics, a step 1004 of locating the third catalyst B in a bottom chamber of the reactor, where the third catalyst B is configured to promote a conversion of a second part of the olefines into additional aromatics, a step 1006 of supplying the methanol feed to the upper chamber of the reactor, and a step 1008 of converting the methanol feed to the aromatics in the top and bottom chambers. The first and second catalysts A1, A2 are mixed together to be in direct contact with each other in the upper chamber.

The step of converting may further include converting the first part of the methanol feed into formaldehyde due to the second catalyst A2, simultaneously converting the second part of the methanol feed into olefins due to the first catalyst A1, reacting in the upper chamber the formaldehyde with the olefines to produce dienes, and reacting the dienes with the first part of the olefines to produce monocyclic aromatics. The method may further include a step of transferring the monocycle aromatics and the second part of olefines to the bottom chamber, and converting the second part of the olefines, due to the third catalyst, into additional monocyclic aromatics in the bottom chamber. In one application, the step of reacting in the upper chamber the formaldehyde with the olefines reacts the entire formaldehyde so that no formaldehyde enters the bottom chamber.

The disclosed embodiments provide a method for generating aromatic hydrocarbons through conversion of methanol via a formaldehyde route with cascade reactions promoted by a multi-component catalyst kit distributed in a dual bed. The embodiments also provide a three-component catalyst kit that achieves this conversion. It should be understood that this description is not intended to limit the invention. On the contrary, the embodiments are intended to cover alternatives, modifications and equivalents, which are included in the spirit and scope of the invention as defined by the appended claims. Further, in the detailed description of the embodiments, numerous specific details are set forth in order to provide a comprehensive understanding of the claimed invention. However, one skilled in the art would understand that various embodiments may be practiced without such specific details.

Although the features and elements of the present embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

1. A catalytic kit for converting a feedstock to aromatics in a dual bed reactor, the catalytic kit comprising:
a first catalyst A1 configured to promote (1) a conversion of a first part of the feedstock into olefins and (2) a conversion of diene and the olefines into the aromatics;
a second catalyst A2 configured to promote a conversion of a second part of the feedstock into formaldehyde; and
a third catalyst B configured to promote a conversion of olefines into additional aromatics,
wherein the first and second catalysts A1, A2 are mixed together to be in direct contact with each other.

2. The catalyst kit of claim 1, wherein the first catalyst A1 is one of Silicon oxide (SiO₂), Magnesium oxide (MgO), ZSM-5, H-ZSM-5, ZSM-11, H-ZSM-11, ZSM-22, H-ZSM-22, MCM-22, H-MCM-22, SSZ-74, H-ZSM-22, SSZ-35, H-SSZ-35 or a mixture thereof.

3. The catalyst kit of claim 2, wherein the catalyst A2 is one of Zinc oxide (ZnO), Vanadium oxide (V₂O₅), Chromium oxide (Cr₂O₃), Magnesium oxide (MgO), Iron oxide (Fe₃O₄), Cobalt oxide (Co₃O₄), Nickel oxide (NiO), Copper oxide (CuO), Gallium oxide (Ga₂O₃), Silver oxide (Ag₂O), Indium oxide (In₂O₃), Molybdenum oxide (MoO₃), Tungsten oxide (WO₃) or a mixture thereof.

4. The catalyst kit of claim 3, wherein the catalyst B is one of Zinc-modified H-ZSM-5 or H-ZSM-11 or H-ZSM-22 or H-MCM-22 or H-ZSM-22 or H-SSZ-35; or Vanadium-modified H-ZSM-5 or H-ZSM-11 or H-ZSM-22 or H-MCM-22 or H-ZSM-22 or H-SSZ-35; or Chromium-modified H-ZSM-5 or H-ZSM-11 or H-ZSM-22 or H-MCM-22 or H-ZSM-22 or H-SSZ-35; or Magnesium-modified H-ZSM-5 or H-ZSM-11 or H-ZSM-22 or H-MCM-22 or H-ZSM-22 or H-SSZ-35; or Iron-modified H-ZSM-5 or H-ZSM-11 or H-ZSM-22 or H-MCM-22 or H-ZSM-22 or H-SSZ-35; or Cobalt-modified H-ZSM-5 or H-ZSM-11 or H-ZSM-22 or H-MCM-22 or H-ZSM-22 or H-SSZ-35; or Nickel-modified H-ZSM-5 or H-ZSM-11 or H-ZSM-22 or H-MCM-22 or H-ZSM-22 or H-SSZ-35; or Copper-modified H-ZSM-5 or H-ZSM-11 or H-ZSM-22 or H-MCM-22 or H-ZSM-22 or H-SSZ-35; or Gallium-modified H-ZSM-5 or H-ZSM-11 or H-ZSM-22 or H-MCM-22 or H-ZSM-22 or H-SSZ-35; or Silver-modified H-ZSM-5 or H-ZSM-11 or H-ZSM-22 or H-MCM-22 or H-ZSM-22 or H-SSZ-35; or Indium-modified H-ZSM-5 or H-ZSM-11 or H-ZSM-22 or H-MCM-22 or H-ZSM-22 or H-SSZ-35; or Molybdenum-modified H-ZSM-5 or H-ZSM-11 or H-ZSM-22 or H-MCM-22 or H-ZSM-22 or H-SSZ-35; or Tungsten-modified H-ZSM-5 or H-ZSM-11 or H-ZSM-22 or H-MCM-22 or H-ZSM-22 or H-SSZ-35.

5. The catalyst kit of claim 1, wherein the first and second catalysts are configured to be placed in a different chamber of the dual bed reactor than the third catalyst.

6. The catalyst kit of claim 5, wherein the feedstock is methanol, the first catalyst A1 is H-ZSM-5 zeolite, the second catalyst A2 is ZnO, and the third catalyst B is Zn-modified H-ZSM-5 zeolite.

7. The catalyst kit of claim 1, wherein a ratio of Si to Al atoms in the first catalyst A1 is between 50 and 1000, including 50, and a ratio of Si to Al atoms in the third catalyst B is between 10 and 50, excluding 50.

8. The catalyst kit of claim 7, wherein a zinc loading of the third catalyst B is between 1 and 5% by mass.

9. The catalyst kit of claim 7, wherein a mass ratio between the first catalyst A1 and the second catalyst A2 is between 0.1 and 10.

10. A reactor configured for converting a feedstock to aromatics, the reactor comprising:
a housing divided into an upper chamber and a bottom chamber by a porous divider;
an inlet configured to receive the feedstock, the inlet-being in fluid communication with the upper chamber;
an outlet configured to discharge the aromatics from the bottom chamber; and
a catalyst kit having first and second catalysts A1 and A2 distributed in the upper chamber and a third catalyst B distributed in the bottom chamber,
wherein the first catalyst A1 is configured to promote (1) a conversion of a first part of the feedstock into olefins, and (2) a conversion of diene and a first part of the olefines into the aromatics, the second catalyst A2 is configured to promote a conversion of a second part of the feedstock into formaldehyde, and the third catalyst B is configured to promote a conversion of a second part of the olefines into additional aromatics, and
wherein the first and second catalysts A1, A2 are mixed together to be in direct contact with each other.

11. The reactor of claim 10, wherein the feedstock is methanol, the first catalyst A1 is H-ZSM-5 zeolite, the second catalyst A2 is ZnO, and the third catalyst B is Zn-modified H-ZSM-5 zeolite.

12. The reactor of claim 11, wherein a ratio of Si to Al atoms in the first catalyst A1 is between 50 and 1000, including 50, and a ratio of Si to Al atoms in the third catalyst B is between 10 and 50, excluding 50.

13. The reactor of claim 12, wherein a zinc loading of the third catalyst B is between 1 and 5% by mass.

14. The reactor of claim 10, wherein a mass ratio between the first catalyst A1 and the second catalyst A2 is between 0.1 and 10.

15. The reactor of claim 10, wherein the divider is made of quartz wool and housing is made of quartz.

16. The reactor of claim 10, wherein the divider occupies between 5 to 10% of the entire reactor, the first and second catalysts occupy about 20 to 40% of the entire reactor, and the third catalyst occupies about 20 to 50% of the entire reactor.

17. A method for converting a methanol feed to aromatics, the method comprising:
receiving a catalyst kit having first and second catalysts A1 and A2, and a third catalyst B, wherein each of the three catalysts is different from the other two catalysts;
loading the first and second catalysts A1 and A2 in an upper chamber of a reactor, wherein the first catalyst A1 is configured to promote (1) a conversion of a first part of the methanol feed into olefins, and (2) a conversion of dienes and a first part of the olefines into the aromatics, and the second catalyst A2 is configured to promote a conversion of a second part of the methanol feed into formaldehyde;
loading the third catalyst B in a bottom chamber of the reactor wherein the third catalyst B is configured to promote a conversion of a second part of the olefines into additional aromatics;
supplying the methanol feed to the upper chamber of the reactor; and
converting the methanol feed to the aromatics in each of the upper and bottom chambers,
wherein the first and second catalysts A1, A2 are mixed together to be in direct contact with each other in the upper chamber.

18. The method of claim 17, wherein the step of converting comprises:
converting the first part of the methanol feed into formaldehyde due to the second catalyst A2;
simultaneously converting the second part of the methanol feed into olefins due to the first catalyst A1;
reacting in the upper chamber the formaldehyde with the olefines to produce dienes; and
reacting the dienes with the first part of the olefines to produce monocyclic aromatics.

19. The method of claim 18, further comprising:
transferring the monocyclic aromatics and the second part of olefines to the bottom chamber; and
converting the second part of the olefines, due to the third catalyst, into additional monocyclic aromatics in the bottom chamber.

20. The method of claim 18, wherein the step of reacting in the upper chamber the formaldehyde with the olefines reacts the entire formaldehyde so that no formaldehyde enters the bottom chamber.
